(19) United States
(12) Patent Application Publication
(10) Pub. No.: US 2017/0336461 A1
(43) Pub. Date: Nov. 23, 2017

(54) INTERNAL TRANSFORMER COMPOSITE-DEFECT FUZZY DIAGNOSTIC METHOD BASED ON GAS DISSOLVED IN OIL

(71) Applicants: State Grid Corporation of China, Beijing (CN); State Grid Hebei Electric Power Research Institute, Hebei (CN); Hebei Electric Power Commissioning Institude, Hebei (CN)

(72) Inventors: Shuguo GAO, Hebei (CN); Hui FAN, Hebei (CN); Zhiyong CHEN, Hebei (CN); Jin PAN, Hebei (CN); Hongliang LIU, Hebei (CN); Jun ZHAO, Hebei (CN)

(21) Appl. No.: 15/324,169

(22) PCT Filed: Aug. 5, 2015
(86) PCT No.: PCT/CN2015/086109

§ 371 Date: May 11, 2017

(30) Foreign Application Priority Data
Feb. 13, 2015 (CN) 2015100773945

(51) Int. Cl. G01R 31/02; G01F 25/00; G01N 33/28; H01F 27/40

(52) U.S. Cl. CPC G01R 31/027; H01F 27/402; G01F 25/00; G01N 33/2841

(57) ABSTRACT

A transformer internal composite defect fuzzy diagnosis method based on gas dissolved in oil, comprising: a step of acquiring monitoring data of volume concentrations of five types of monitored feature gas; a step of determining ratio codes; a step of modifying a three-ratio method; a step of fuzzifying a boundary range; a step of calculating probabilities of the ratio codes; a step of calculating a probability of occurrence of each defect fault; and finally obtaining a fault type of a transformer. The method has the beneficial effects that: the method is simple and easy to achieve, and particularly suitable for being applied to an on-line transformer state monitoring system; based on a concept of fuzzy logic, diagnosis of composite defects of the transformer under a complicated state and evaluation of the degree of severity can be achieved, and the problem of sudden change caused by criterion boundary absolutisation can be effectively avoided; and multi-feature information such as an attention value and a ratio of the gas dissolved in the oil are merged and analysed, thereby effectively improving the diagnosis reliability.

### FIELD OF THE INVENTION

The present invention relates to the technical field of power transformer fault diagnosis, in particular to a fuzzy diagnosis method for transformer internal composite defect based on gas dissolved in oil.

### BACKGROUND OF THE INVENTION

Power transformers are important devices in electric power systems and are of great significance for safe and reliable operation of the electric grid. Routine test of power transformers is an important means for detecting potential hazards in the transformers and avoid outburst of accidents. Among the tests, transformer oil chromatogram test is a very effective test and includes rich information on insulation state of the device, and can be used to find defects in the transformer such as internal discharge, local overheat, and moistened insulation, etc.; hence, transformer oil chromatogram test is widely applied in electric power systems. Accurate diagnosis of internal defects in transformers is helpful for judging the positions and types of the defects in the devices, and is always a key subject in the research in the art. Based on that, a scientific overhaul strategy can be established, and thereby the efficiency of equipment operation, maintenance and overhaul can be greatly improved, and the power distribution reliability of the electric grid can be improved. At present, classical transformer oil chromatogram analysis methods mainly include characteristic gas method, Rogers ratio method, IEC three-ratio method, Dewey triangular chart method, and modified three-ratio method, etc. Among those methods, the characteristic gas method is a method for rating the defect according to the concentration of characteristic gases and total hydrocarbon concentration, and is only suitable for qualitatively judging whether there is any defect or not. The Rogers four-ratio method is developed on the basis of Doerenburg five-ratio method, and utilizes gas ratios as criteria for judging the types of transformer defects, like the IEC three-ratio method. Such kind of ratio methods only make sense for the ratios of defective transformer, but may result in misjudgement under normal conditions; in addition, these methods may have problems in actual application, for example, there is no corresponding ratio code, the boundaries of criteria are absolutized, and composite defects cannot be diagnosed accurately, etc. The modified three-ratio method proposed in the Chinese Standard GB/T7252-2001 is developed on the basis of the IEC three-ratio method by correcting corresponding codes according to the statistical analysis result of transformer data in China, but it still utilize gas ratios as criteria for judging the types of transformer defects. The Dewey triangular chart method is a method that differentiate the types of defects on the basis of the triangular chart coordinates of gas ratio distribution, in which each type of defects corresponds to a certain region; though this method solves the problem that there is no corresponding code in the ratio method, but still have other problems, for example, the boundaries are absolutized and composite defects cannot be diagnosed accurately.

In summary, the diagonisis characteristic criteria used in different oil chromatogram diagnosis methods for transformer defects only rely on simplex characteristic information, such as characteristic gas type, gas concentration or gas ratio, the boundaries of diagnostic criteria are too absolutized, and the diagnosis result cannot reveal the severities or probabilities of occurrence of the defects. In actual application, the transformer defects are complex, and are usually composite defects; consequently, such defects cannot be identified with existing diagnosis methods. Therefore, it is necessary to improve the existing oil chromatogram diagnosis methods for transformer internal defects.

### CONTENTS OF THE INVENTION

The technical problem to be solved by the present invention is to provide a fuzzy diagnosis method for transformer internal composite defect based on gas dissolved in oil, which can effectively solve the problem that the criteria boundaries are absolutized and composite defects cannot be diagnosed with conventional analysis method based on gas dissolved in oil, can comprehensively utilize the information of a variety of characteristic quantities, and can effectively improve the reliability of defect and fault diagnosis.

To solve the above-mentioned technical problem, the present invention employs the following technical scheme: a fuzzy diagnosis method for transformer internal composite defect based on gas dissolved in oil, comprising the following steps:

(I) Acquiring monitoring data of volumetric concentration of five types of monitored characteristic gases, i.e., hydrogen, methane, ethane, ethylene and acetylene; calculating the sum of the volumetric concentrations of methane, ethane, ethylene and acetylene (i.e., volumetric concentration of total hydrocarbons) from the monitoring data; judging whether the monitoring data of the five types of characteristic gases or the volumetric concentration of total hydrocarbons exceeds an alert value, which is selected as per the Chinese Standard GB/T7252-2001; if the monitoring data or the volumetric concentration of total hydrocarbons exceeds the alert value, further diagnosis is required; in that case, going to step (II); otherwise judging that the transformer has no defect or fault, if the monitoring data and the volumetric concentration of total hydrocarbons are normal;

(II) Determining ratio codes:

First, the ratios are set as follows:

$${r}_{1} = \frac{{c}_{1} ( {C}_{2} {H}_{2} )}{{c}_{2} ( {C}_{2} {H}_{4} )} , \\ {r}_{2} = \frac{{c}_{3} ( {CH}_{4} )}{{c}_{4} ( {H}_{2} )} , \\ {r}_{3} = \frac{{c}_{2} ( {C}_{2} {H}_{4} )}{{c}_{5} ( {C}_{2} {H}_{6} )} , \\ {r}_{4} = \frac{{c}_{2} ( {C}_{2} {H}_{4} )}{{c}_{5} ( {C}_{2} {H}_{6} )} \times \frac{{c}_{2} ( {C}_{2} {H}_{4} )}{{c}_{1} ( {C}_{2} {H}_{2} )} = {r}_{3} \times \frac{{c}_{2} ( {C}_{2} {H}_{4} )}{{c}_{1} ( {C}_{2} {H}_{2} )}$$

wherein, c₁(C₂H₂), c₂(C₂H₄), c₃(CH₄), c₄(H₂) and c₅(C₂H₆) respectively represent the volumetric concentration of five types of characteristic gases (acetylene, ethylene, methane, hydrogen and ethane), in unit of μL/L;

Then, the ratio codes are determined according to the following rules:

If r₁<0.1, the ratio code of r₁ is 0; if 0.1≦r₁<1, the ratio code of r₁ is 1; if 5.1r₁<3, the ratio code of r₁ is 1; if r₁ the ratio code of r₁ is 2;

If r₂<0.1, the ratio code of r₂ is 1; if 0.1≦r₂<1, the ratio code of r₂ is 0; if 1≦r₂<3, the ratio code of r₂ is 2; if the ratio code of r₂ is 2;

If r₃<0.1, the ratio code of r₃ is 0; if 0.1r₃<1, the ratio code of r₃ is 0; if 1.5r₃<3, the ratio code of r₃ is 1; if r₃≧3, the ratio code of r₃ is 2;

If r₄≦1.5, the ratio code of r₄ is 0; if r₄>1.5, the ratio code of r₄ is 1;

(III) Correcting the method for determining the types of transformer defects or faults on the basis of three ratios as specified in the Chinese Standard GB/T7252-2001:

Based on the types of transformer defects or faults corresponding to the three ratio codes specified in the Chinese Standard GB/T7252-2001, a ratio code 011 corresponding to the defect or fault type of partial discharge is added;

A fourth ratio r₄ is added on the basis of the three ratio codes; for the type of defect or fault with ratio code 101 diagnosed with the three-ratio method, if r₄≦1.5, the transformer is judged as having a spark discharge defect or fault; if r₄>1.5, the transformer is judged as having an arc discharge defect or fault;

Thus, a method for judging the types of transformer defects or faults according to ratio codes is obtained as follows:

If the ratio code of r₁ is 0, the ratio code of r₂ is 1, the ratio code of r3 is 0, 1 or 2, and the ratio code of r₄ is 0 or 1, the type of transformer defect or fault is partial discharge;

If the ratio code of r₁ is 0, the ratio code of r₂ is 0, the ratio code of r₃ is 1, and the ratio code of r₄ is 0 or 1, the type of transformer defect or fault is low-temperature overheat lower than 300□;

If the ratio code of r₁ is 0, the ratio code of r₂ is 2, the ratio code of r₃ is 0, and the ratio code of r₄ is 0 or 1, the type of transformer defect or fault is low-temperature overheat lower than 300□;

If the ratio code of r₁ is 0, the ratio code of r₂ is 2, the ratio code of r₃ is 1, and the ratio code of r₄ is 0 or 1, the type of transformer defect or fault is 300-700□ moderate-temperature overheat;

If the ratio code of r₁ is 0, the ratio code of r₂ is 0 or 2, the ratio code of r₃ is 2, and the ratio code of r₄ is 0 or 1, the type of transformer defect or fault is high-temperature overheat higher than 700□;

If the ratio code of r₁ is 2, the ratio code of r₂ is 0, 1 or 2, the ratio code of r₃ is 0, 1 or 2, and the ratio code of r₄ is 0 or 1, the type of transformer defect or fault is spark discharge;

If the ratio code of r₁ is 1, the ratio code of r₂ is 0, the ratio code of r₃ is 1, and the ratio code of r₄ is 0, the type of transformer defect or fault is spark discharge;

If the ratio code of r₁ is 1, the ratio code of r₂ is 0, the ratio code of r₃ is 1, and the ratio code of r₄ is 1, the type of transformer defect or fault is arc discharge;

If the ratio code of r₁ is 1, the ratio code of r₂ is 0, 1 or 2, the ratio code of r₃ is 0 or 2, and the ratio code of r₄ is 0 or 1, the type of transformer defect or fault is arc discharge;

If the ratio code of r₁ is 1, the ratio code of r₂ is 1 or 2, the ratio code of r₃ is 1, and the ratio code of r₄ is 0 or 1, the type of transformer defect or fault is arc discharge;

(IV) Blurring the boundary ranges of the ratios r₁, r₂, r₃ and r₄ with a semi-Cauchy rising/falling function, and representing the rising edges and falling edges of the boundaries with the semi-Cauchy rising/falling function as follows:

$${μ}_{d} ( r ) = \{ \begin{matrix}1 & , r \leq A \\ \frac{1}{1 + {( \frac{A - r}{a} )}^{2}} & , others\end{matrix} \\ {μ}_{a} ( r ) = \{ \begin{matrix}1 & , r \geq A \\ \frac{1}{1 + {( \frac{A - r}{a} )}^{2}} & , others\end{matrix}$$

wherein, μ_{d}(r) is a falling edge function; μₐ(r) is a rising edge function; A is a boundary parameter; a is a distribution parameter; the values of A and a are as follows:

The rising edge boundary parameter of r₁ is 0.08, and the corresponding distribution parameter is 0.01

The falling edge boundary parameter of r₁ is 3.1, and the corresponding distribution parameter is 0.1;

The rising edge boundary parameter of r₂ is 0.06, and the corresponding distribution parameter is 0.02;

The falling edge boundary parameter of r₂ is 0.6, and the corresponding distribution parameter is 0.2;

The rising edge boundary parameter of r₃ is 0.8, and the corresponding distribution parameter is 0.1;

The falling edge boundary parameter of r₃ is 3.6, and the corresponding distribution parameter is 0.3;

The boundary parameter of r₄ is 1.43, and the corresponding distribution parameter is 0.1;

(V) Obtaining the probabilities of the cases that the ratio codes of the ratios r₁, r₂ and r₃ are 0, 1 and 2 respectively and the probabilities of the cases that the ratio code of r₄ is 0 or 1 respectively, with the semi-Cauchy rising/falling function; the expressions are as follows:

Probability f-code0(r₁) of the case that the ratio code of r₁ is 0:

$$f \text{-} code 0 ( {r}_{1} ) = \{ \begin{matrix}1 & ( {r}_{1} \leq 0.08 ) \\ \frac{1}{1 + {( \frac{0.08 - {r}_{1}}{0.01} )}^{2}} & ( {r}_{1} > 0.08 )\end{matrix}$$

Probability f-code1(r₁) of the case that the ratio code of r₁ is 1:

$$f \text{-} code 1 ( {r}_{1} ) = \{ \begin{matrix}\frac{1}{1 + {( \frac{0.08 - {r}_{1}}{0.01} )}^{2}} & ( {r}_{1} < 0.08 ) \\ 1 & ( 0.08 \leq {r}_{1} \leq 3.1 ) \\ \frac{1}{1 + {( \frac{3.1 - {r}_{1}}{0.1} )}^{2}} & ( {r}_{1} > 3.1 )\end{matrix}$$

Probability f-code2(r₁) of the case that the ratio code of r₁ is 2:

$$f \text{-} code 2 ( {r}_{1} ) = \{ \begin{matrix}\frac{1}{1 + {( \frac{3.1 - {r}_{1}}{0.1} )}^{2}} & ( {r}_{1} < 3.1 ) \\ 1 & ( {r}_{1} \geq 3.1 )\end{matrix}$$

Probability f-code0(r₂) of the case that the ratio code of r₂ is 0:

$$f \text{-} code 0 ( r 2 ) = \{ \begin{matrix}\frac{1}{1 + {( \frac{0.06 - {r}_{2}}{0.02} )}^{2}} & ( {r}_{2} < 0.06 ) \\ 1 & ( 0.06 \leq {r}_{2} \leq 0.6 ) \\ \frac{1}{1 + {( \frac{0.6 - {r}_{2}}{0.2} )}^{2}} & ( {r}_{2} > 0.6 )\end{matrix}$$

Probability f-code1(r₂) of the case that the ratio code of r₂ is 1:

$$f \text{-} code 1 ( r 2 ) = \{ \begin{matrix}1 & ( {r}_{2} \leq 0.06 ) \\ \frac{1}{1 + {( \frac{0.06 - {r}_{2}}{0.02} )}^{2}} & ( {r}_{2} > 0.06 )\end{matrix}$$

Probability f-code2(r₂) of the case that the ratio code of r₂ is 2:

$$f \text{-} code 2 ( {r}_{2} ) = \{ \begin{matrix}\frac{1}{1 + {( \frac{0.6 - {r}_{2}}{0.2} )}^{2}} & ( {r}_{2} < 0.6 ) \\ 1 & ( {r}_{2} \geq 0.6 )\end{matrix}$$

Probability f-code0(r₃) of the case that the ratio code of r₃ is 0:

$$f \text{-} code 0 ( {r}_{3} ) = \{ \begin{matrix}1 & ( {r}_{3} \leq 0.8 ) \\ \frac{1}{1 + {( \frac{0.8 - {r}_{3}}{0.1} )}^{2}} & ( {r}_{3} > 0.8 )\end{matrix}$$

Probability f-code1(r₃) of the case that the ratio code of r₃ is 1:

$$f \text{-} code 1 ( {r}_{3} ) = \{ \begin{matrix}\frac{1}{1 + {( \frac{0.6 - {r}_{3}}{0.1} )}^{2}} & ( {r}_{3} < 0.8 ) \\ 1 & ( 0.8 \leq {r}_{3} \leq 3.6 ) \\ \frac{1}{1 + {( \frac{3.6 - {r}_{3}}{0.3} )}^{2}} & ( {r}_{3} > 3.6 )\end{matrix}$$

Probability f-code2(r₃) of the case that the ratio code of r₃ is 2:

$$f \text{-} code 2 ( {r}_{3} ) = \{ \begin{matrix}\frac{1}{1 + {( \frac{3.6 - {r}_{3}}{0.3} )}^{2}} & ( {r}_{3} > 3.6 ) \\ 1 & ( 0.8 \leq {r}_{3} \leq 3.6 )\end{matrix}$$

Probability f-code0(r₄) of the case that the ratio code of r₄ is 0:

$$f \text{-} code 0 ( {r}_{4} ) = \{ \begin{matrix}1 & ( {r}_{4} \leq 1.43 ) \\ \frac{1}{1 + {( \frac{1.43 - {r}_{4}}{0.1} )}^{2}} & ( {r}_{4} > 1.43 )\end{matrix}$$

Probability f-code1(r₄) of the case that the ratio code of r₄ is 1:

$$f \text{-} code 1 ( {r}_{4} ) = \{ \begin{matrix}\frac{1}{1 + {( \frac{1.43 - {r}_{4}}{0.1} )}^{2}} & ( {r}_{4} < 1.43 ) \\ 1 & ( {r}_{4} \geq 1.43 )\end{matrix}$$

(VI) Representing the probabilities of ratio codes with maximum value logic and minimum value logic, and thereby obtaining a fuzzy multi-valued form of the diagnostic result of the types of transformer defects or faults; the probabilities of the types of transformer defects or faults are as follows:

f(partial discharge)=min[f-code0(r₁), f-code1(r₂)];

f(low-temperature overheat)=max{min[f-code0(r₁), f-code0(r₂), f-code1(r₃)], min[f-code0(r₁), f-code2(r₂), f-code0(r3)]};

f(moderate-temperature overheat)=min[f-code0(r₁), f-code2(r₂), f-code1(r3)];

f(high-temperature overheat)=max{min[f-code0(r₁), f-code0(r₂), f-code2(r₃)], min[f-code0(r₁), f-code2(r2), f-code2(r₃)]};

f(spark discharge)=max{f-code2(r₁), min[f-code 1(r₁), f-code0(r₂), f-code1(r₃), f-code0(r₄)]};

f(arc discharge)=max{min[f-code 1(r₁), f-code0(r₂), f-code1(r₃), f-code1(r₄)], min[f-code1(r₁), f-code0(r₃)], min[f-code 1(r₁), f-code2(r₃)], min [f-code1(r₁), f-code1(r₂), f-code1(r₃)], min[f-code 1(r₁), f-code2(r₂), f-code 1(r₃)]}.

The beneficial effects of the present invention include: the present invention is simple and easy to implement, and is especially suitable for the application of online transformer state monitoring systems; based on a concept of fuzzy logic, diagnosis of composite defects of the transformer in a complex state and evaluation of the degree of severity can be achieved, and the problem of sudden change caused by criterion boundary absolutization can be effectively avoided; and a variety of characteristic information, such as alert value and ratio of the gas dissolved in the oil, are merged and analyzed, and thereby the reliability of diagnosis is improved effectively.

### DESCRIPTION OF THE DRAWINGS

FIG. 1 is a diagnostic flow chart according to the present invention;

FIG. 2 shows the fuzzy boundary when the ratio code of the ratio r₃ is 2.

### EMBODIMENTS

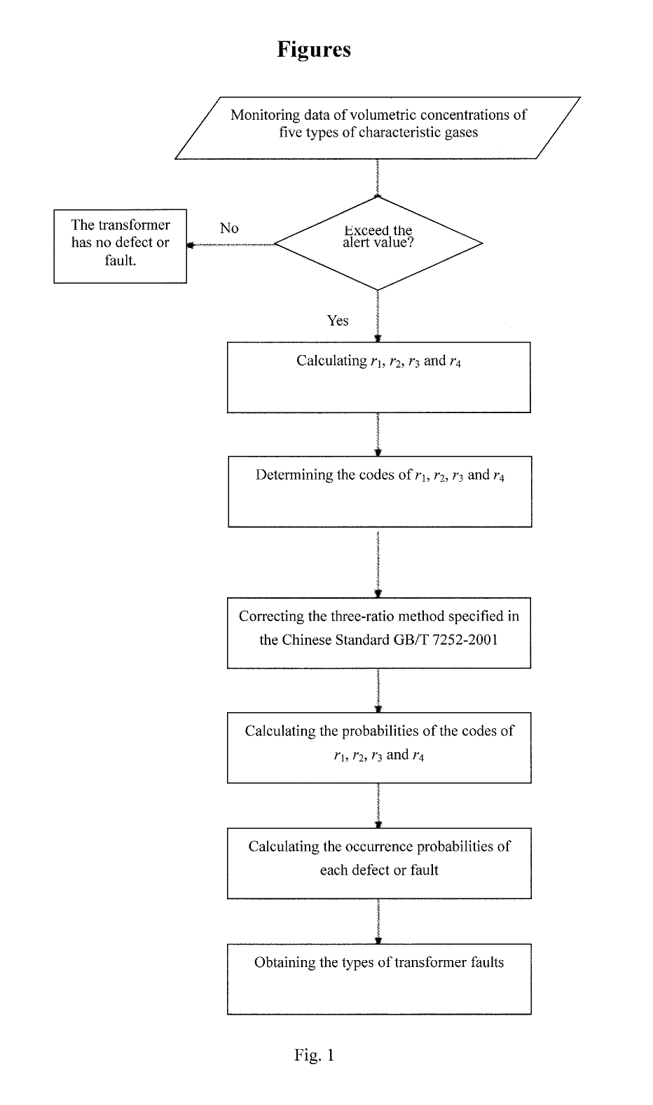

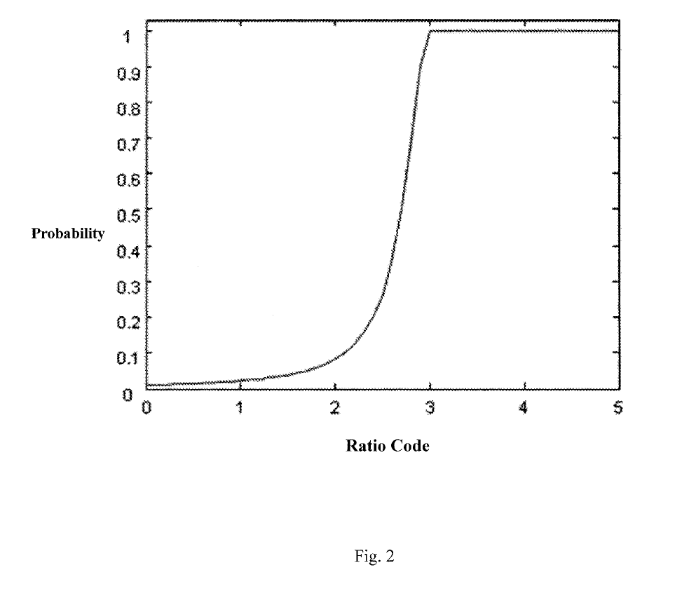

Hereunder the present invention will be further described in an example, with reference to FIGS. 1-2.

The implementation steps of this example are as follows:

(I) Acquiring monitoring data of volumetric concentrations of five types of monitored characteristic gases, i.e., hydrogen, methane, ethane, ethylene and acetylene; calculating the sum of the volumetric concentrations of methane, ethane, ethylene and acetylene (i.e., volumetric concentration of total hydrocarbons) from the monitoring data; judging whether the monitoring data of the five types of characteristic gases or the volumetric concentration of total hydrocarbons exceeds an alert value; the alert value is selected as per the Chinese Standard GB/T7252-2001 “Guide to the Analysis and Diagnosis of Gases Dissolved in Transformer Oil”; if the monitoring data or the volumetric concentration of total hydrocarbons exceeds the alert value, further diagnosis is required; in that case, going to step (II); otherwise judging that the transformer has no defect or fault, if the monitoring data and the volumetric concentration of total hydrocarbons are normal;

(II) Determining ratio codes:

First, the ratios are set as follows:

$${r}_{1} = \frac{{c}_{1} ( {C}_{2} {H}_{2} )}{{c}_{2} ( {C}_{2} {H}_{4} )} , \\ {r}_{2} = \frac{{c}_{3} ( {CH}_{4} )}{{c}_{4} ( {H}_{2} )} , \\ {r}_{3} = \frac{{c}_{2} ( {C}_{2} {H}_{4} )}{{c}_{5} ( {C}_{2} {H}_{6} )} , \\ {r}_{4} = \frac{{c}_{2} ( {C}_{2} {H}_{4} )}{{c}_{5} ( {C}_{2} {H}_{6} )} \frac{{c}_{2} ( {C}_{2} {H}_{4} )}{{c}_{1} ( {C}_{2} {H}_{2} )} = {r}_{3} \frac{{c}_{2} ( {C}_{2} {H}_{4} )}{{c}_{1} ( {C}_{2} {H}_{2} )} i$$

Where, c₁(C₂H₂), c₂(C₂H₄), c₃(CH₄), c₄(H₂), and c₅(C₂H₆) respectively represent the volumetric concentration of five types of characteristic gases (acetylene, ethylene, methane, hydrogen and ethane), in unit of μL/L;

Then, the ratio codes are determined according to the rules shown in Table 1:

TABLE 1
Rules for Determining Ratio Codes

| Ratio Range | Ratio Code | | | Ratio Range | Ratio Code |
|---|---|---|---|---|---|
| r₁, r₂ or r₃ | r₁ | r₂ | r₃ | r₄ | r₄ |
| <0.1 | 0 | 1 | 0 | ≦1.5 | 0 |
| ≧0.1~<1 | 1 | 0 | 0 | | |
| ≧1~<3 | 1 | 2 | 1 | >1.5 | 1 |
| ≧3 | 2 | 2 | 2 | | |

wherein, the ratio codes of r₁, r₂ and r₃ are obtained according to the ratio code rules specified in the Chinese Standard GB/T 7252-2001 “Guide to the Analysis and Diagnosis of Gases Dissolved in Transformer Oil”; a ratio r₄ and a r₄ ratio code rule are added on the basis of the ratio code rules specified in the Chinese Standard GB/T 7252-2001 “Guide to the Analysis and Diagnosis of Gases Dissolved in Transformer Oil”;

(III) Correcting the three-ratio method for judging the types of transformer defects or faults specified in the Chinese Standard GB/T 7252-2001 “Guide to the Analysis and Diagnosis of Gases Dissolved in Transformer Oil” on the basis of analysis of 728 typical real fault cases of the State Grid Corporation of China; thus, rules for judging the types of transformer defects or faults based on ratio codes are obtained, as shown in Table 2.

A fourth ratio r₄ is added on the basis of three ratio codes; for the type of defect or fault with ratio code 101 diagnosed with the three-ratio method, if r₄≦1.5, the transformer is judged as having a spark discharge defect or fault; if r₄>1.5, the transformer is judged as having an arc discharge defect or fault;

Based on the codes for transformer faults and defects specified in the Chinese Standard GB/T 7252-2001 “Guide to the Analysis and Diagnosis of Gases Dissolved in Transformer Oil”, a ratio code 011 corresponding to partial discharge fault or defect is added.

TABLE 2
Method for Judging the Types of Transformer Defects or Faults
according to Ratio Codes

| Combinations of Ratio Codes | | | | |
|---|---|---|---|---|
| r₁ | r₂ | r₃ | r₄ | Type of Defect or Fault |
| 0 | 1 | 0, 1, 2 | 0, 1 | Local discharge |
| | 0 | 1 | 0, 1 | Low-temperature overheat lower |
| | 2 | 0 | 0, 1 | than 300□ |
| | 2 | 1 | 0, 1 | 300-700° C. |
| | | | | moderate-temperature overheat |
| | 0, 2 | 2 | 0.1 | High-temperature overheat higher |
| | | | | than 700□ |
| 2 | 0, 1, 2 | 0, 1, 2 | 0, 1 | Spark discharge |
| 1 | 0 | 1 | 0 | |
| | | | 1 | Arc discharge |
| | 0, 1, 2 | 0, 2 | 0, 1 | |
| | 1, 2 | 1 | | |

(IV) Blurring the boundaries of the codes in Table 1 with a semi-Cauchy rising/falling function, and representing the rising edges and falling edges of the boundaries with the semi-Cauchy rising/falling function, in order to change the either-or absolutized boundary judgment; then, obtaining the probabilities of the cases that the ratio codes of the ratios r₁, r₂ and r₃ are 0, 1 and 2 respectively (represented by f-code0(rᵢ), f-code1(rᵢ), f-code2(rᵢ) respectively) and the probabilities of the cases that the ratio code of r4 is 0 or 1 respectively, with the semi-Cauchy rising/falling function. For example, the probability of the case that the ratio code of the ratio r₃ is 2 is represented by f-code2(r₃), and the fuzzy boundary is represented by the semi-Cauchy rising edge function, as shown in FIG. 2.

The boundary ranges of the ratios r₁, r₂, r₃ and r₄ are blurred with a semi-Cauchy rising/falling function, and the rising edges and falling edges of the boundaries are represented with the semi-Cauchy rising/falling function as follows:

$${μ}_{d} ( r ) = \{ \begin{matrix}1 & , r \leq A \\ \frac{1}{1 + {( \frac{A - r}{a} )}^{2}} & , others\end{matrix} \\ {μ}_{a} ( r ) = \{ \begin{matrix}1 & , r \geq A \\ \frac{1}{1 + {( \frac{A - r}{a} )}^{2}} & , others\end{matrix}$$

wherein, μ_{d}(r) is a falling edge function; μₐ(r) is a rising edge function; A is a boundary parameter; a is a distribution parameter; the values of A and a are optimal values obtained through verification of the data of 728 typical real fault cases of the State Grid Coporation of China, as shown in Table 3.

TABLE 3
Boundary Parameter A and Distribution Parameter a

| A₁(r₁) | A₂(r₁) | A₁(r₂) | A₂(r₂) | A₁(r₃) | A₂(r₃) | A(r₄) |
|---|---|---|---|---|---|---|
| 0.08 | 3.1 | 0.06 | 0.6 | 0.8 | 3.6 | 1.43 |
| a₁(r₁) | a₂(r₁) | a₁(r₂) | a₂(r₂) | a₁(r₃) | a₂ (r₃) | a(r₄) |
| 0.01 | 0.1 | 0.02 | 0.2 | 0.1 | 0.3 | 0.1 |

In the Table 3:

The rising edge boundary parameter of r₁, A₁(r₁), is 0.08, and the corresponding distribution parameter a₁(r₁) is 0.01;

The falling edge boundary parameter of r₁, A₂(r₁), is 3.1, and the corresponding distribution parameter a₂(r₁) is 0.1;

The rising edge boundary parameter of r₂, A₁(r₂), is 0.06, and the corresponding distribution parameter a₁(r₂) is 0.02;

The falling edge boundary parameter of r₂, A₂(r₂), is 0.6, and the corresponding distribution parameter a₂(r₂) is 0.2;

The rising edge boundary parameter of r₃, A₁(r₃), is 0.8, and the corresponding distribution parameter a₁(r₃) is 0.1;

The falling edge boundary parameter of r₃, A₂(r₃), is 3.6, and the corresponding distribution parameter a₂(r₃) is 0.3;

The boundary parameter of r₄, A(r₄), is 1.43, and the corresponding distribution parameter a(r₄) is 0.1;

(V) Obtaining the probabilities of the cases that the ratio codes of the ratios r₁, r₂ and r₃ are 0, 1 and 2 respectively and the probabilities of the cases that the ratio code of r₄ is 0 or 1 respectively, with the semi-Cauchy rising/falling function; the expressions are as follows:

Probability f-code0(r₁) of the case that the ratio code of r₁ is 0:

$$f \text{-} code 0 ( {r}_{1} ) = \{ \begin{matrix}1 & ( {r}_{1} \leq 0.08 ) \\ \frac{1}{1 + {( \frac{0.08 - {r}_{1}}{0.01} )}^{2}} & ( {r}_{1} > 0.08 )\end{matrix} \qquad ( expression 1 )$$

Probability f-code1(r₁) of the case that the ratio code of r₁ is 1:

$$f \text{-} code 1 ( {r}_{1} ) = \{ \begin{matrix}\frac{1}{1 + {( \frac{0.08 - {r}_{1}}{0.01} )}^{2}} & ( {r}_{1} \leq 0.08 ) \\ 1 & ( 0.08 \leq {r}_{1} \leq 3.1 ) \\ \frac{1}{1 + {( \frac{3.1 - {r}_{1}}{0.1} )}^{2}} & ( {r}_{1} > 3.1 )\end{matrix} \qquad ( expression 2 )$$

Probability f-code2(r₁) of the case that the ratio code of r₁ is 2:

$$f \text{-} code 2 ( {r}_{1} ) = \{ \begin{matrix}\frac{1}{1 + {( \frac{3.1 - {r}_{1}}{0.1} )}^{2}} & ( {r}_{1} < 3.1 ) \\ 1 & ( {r}_{1} \geq 3.1 )\end{matrix} \qquad ( expression 3 )$$

Probability f-code0(r₂) of the case that the ratio code of r₂ is 0:

$$f \text{-} code 0 ( r 2 ) = \{ \begin{matrix}\frac{1}{1 + {( \frac{0.06 - {r}_{2}}{0.02} )}^{2}} & ( {r}_{2} < 0.06 ) \\ 1 & ( 0.06 \leq {r}_{2} \leq 0.6 ) \\ \frac{1}{1 + {( \frac{0.6 - {r}_{2}}{0.2} )}^{2}} & ( {r}_{2} > 0.6 )\end{matrix} \qquad ( expression 4 )$$

Probability f-code1(r₂) of the case that the ratio code of r₂ is 1:

$$f \text{-} code 1 ( r 2 ) = \{ \begin{matrix}1 & ( {r}_{2} \leq 0.06 ) \\ \frac{1}{1 + {( \frac{0.06 - {r}_{2}}{0.02} )}^{2}} & ( {r}_{2} > 0.06 )\end{matrix} \qquad ( expression 5 )$$

Probability f:code2(r₂) of the case that the ratio code of r₂ is 2:

$$f \text{-} code 2 ( {r}_{2} ) = \{ \begin{matrix}\frac{1}{1 + {( \frac{0.6 - {r}_{2}}{0.2} )}^{2}} & ( {r}_{2} < 0.6 ) \\ 1 & ( {r}_{2} \geq 0.6 )\end{matrix} \qquad ( expression 6 )$$

Probability f-code0(r₃) of the case that the ratio code of r₃ is 0:

$$f \text{-} code 0 ( {r}_{3} ) = \{ \begin{matrix}1 & ( {r}_{3} \leq 0.8 ) \\ \frac{1}{1 + {( \frac{0.8 - {r}_{3}}{0.1} )}^{2}} & ( {r}_{3} > 0.8 )\end{matrix} \qquad ( expression 7 )$$

Probability f-code1 (r₃) of the case that the ratio code of r₃ is 1:

$$f \text{-} code 1 ( {r}_{3} ) = \{ \begin{matrix}\frac{1}{1 + {( \frac{0.6 - {r}_{3}}{0.1} )}^{2}} & ( {r}_{3} < 0.8 ) \\ 1 & ( 0.8 \leq {r}_{3} \leq 3.6 ) \\ \frac{1}{1 + {( \frac{3.6 - {r}_{3}}{0.3} )}^{2}} & ( {r}_{3} > 3.6 )\end{matrix} \qquad ( expression 8 )$$

Probability f-code2(r₃) of the case that the ratio code of r₃ is 2:

$$f \text{-} code 2 ( {r}_{3} ) = \{ \begin{matrix}\frac{1}{1 + {( \frac{3.6 - {r}_{3}}{0.3} )}^{2}} & ( {r}_{3} > 3.6 ) \\ 1 & ( 0.8 \leq {r}_{3} \leq 3.6 )\end{matrix} \qquad ( expression 9 )$$

Probability f-code0(r₄) of the case that the ratio code of r₄ is 0:

$$f \text{-} code 0 ( {r}_{4} ) = \{ \begin{matrix}1 & ( {r}_{4} \leq 1.43 ) \\ \frac{1}{1 + {( \frac{1.43 - {r}_{4}}{0.1} )}^{2}} & ( {r}_{4} > 1.43 )\end{matrix} \qquad ( expression 10 )$$

Probability f-code1(r₄) of the case that the ratio code of r₄ is 1:

$$f - code 1 ( {r}_{4} ) = \{ \begin{matrix}\frac{1}{1 + {( \frac{1.43 - {r}_{4}}{0.1} )}^{2}} & ( {r}_{4} < 1.43 ) \\ 1 & ( {r}_{4} \geq 1.43 )\end{matrix} \qquad ( expression 11 )$$

(VI) Replacing the 0 logic and 1 logic in the ratio code judgement rule with minimum value logic and maximum value logic respectively, carrying out defect and fault diagnosis according to the correspondence between the ratio codes and the types of transformer defects or faults, and representing the result of diagnosis in a fuzzy multi-value form; the result is represented in the form of probability, the result of diagnosis is the probability of occurrence of defect, i.e., severity; the sum of the probabilities of all kinds of faults is 1; the probabilities of the ratio codes are represented by maximum value logic and minimum value logic, and the probabilities of the faults are:

f(partial discharge)=min[f-code0(r₁), f-code1(r₂)]; (expression 12)

f(low-temperature overheat)=max{min[f-code0(r₁), f-code0(r₂), f-code1(r₃)], min[f-code0(r₁), f-code2(r₂), f-code0(r3)]}; (expression 13)

f(moderate-temperature overheat)=min[f-code0(r₁), f-code2(r₂), f-code1(r3)];

f(high-temperature overheat)=max{min[f-code0(r₁), f-code0(r₂), f-code2(r₃)], min[f-code0(r₁), f-code2(r2), f-code2(r₃)]}; (expression 14)

f(spark discharge)=max{f-code2(r₁), min[f-code 1(r₁), f-code0(r₂), f-code1(r₃), f-code0(r₄)]}; (expression 15)

f(arc discharge)=max{min[f-code 1(r₁), f-code0(r₂), f-code1(r₃), f-code1(r₄)], min[f-code1(r₁), f-code0(r₃)], min[f-code 1(r₁), f-code2(r₃)], min [f-code1(r₁), f-code1(r₂), f-code1(r₃)], min[f-code 1(r₁), f-code2(r₂), f-code 1(r₃)]}. (expression 16)

### EXAMPLE 1

The oil chromatogram test data of a transformer (volumetric concentrations of five characteristic gases and total hydrocarbons, in unit of μL/L) is listed in Table 4.

TABLE 4
Oil Chromatogram Test Data of a Transformer

| | | | | | | c_{z}(total |
|---|---|---|---|---|---|---|
| Test Date | c₄(H₂) | c₃(CH₄) | c₅(C₂H₆) | c₂(C₂H₄) | c₁(C₂H₂) | hydrocarbons) |
| Apr. 26, 2012 | 31.33 | 10.52 | 1.98 | 4.01 | 6.09 | 22.60 |

As can be seen from Table 4, the volumetric concentration of acetylene exceeds the alert value, thus the transformer is abnormal.
1. Calculating the four ratios respectively:

$${r}_{1} = \frac{{c}_{1} ( {C}_{2} {H}_{2} )}{{c}_{2} ( {C}_{2} {H}_{4} )} = 1.52 , \\ {r}_{2} = \frac{{c}_{3} ( {CH}_{4} )}{{c}_{4} ( {H}_{2} )} = 2.03 , \\ {r}_{3} = \frac{{c}_{2} ( {C}_{2} {H}_{4} )}{{c}_{5} ( {C}_{2} {H}_{6} )} = 2.03 , \\ {r}_{4} = \frac{{c}_{1} ( {C}_{2} {H}_{4} )}{{c}_{5} ( {C}_{2} {H}_{6} )} \times \frac{{c}_{2} ( {C}_{2} {H}_{4} )}{{c}_{1} ( {C}_{2} {H}_{2} )} = 1.33 ;$$

2. Calculating with the expressions 1 to 11, to obtain the probabilities of the ratio codes of the four ratios:

f-code0(r₁)=0; f-code1(r₁)=1; f-code2(0=0.004;

f-code0(r₂)=1; f-code1(r₂)=0.0051; f-code2(r₂)=0.37;

f-code0(r₃)=0.00657; f-code l(r₃)=1; f-code2(r₃)=0.035;

f-code0(r₄)=-1; f-code2(r₄)=0.5;
3. Calculating with the expression 12 to 16, to obtain the probabilities of the faults:

f(partial discharge)=0%;

f(low-temperature overheat)=0%;

f(moderate-temperature overheat)=0%;

f(high-temperature overheat)=0%;

f(spark discharge)=66.7%;

f(arc discharge)=33.3%;
4. Diagnosing the transformer faults

It is judged from the above probabilities of faults, the transformer has spark discharge fault and arc discharge fault.

While the above embodiments are only preferred embodiments of the present invention, the feasible embodiments of the present invention are not exhausted. Those having ordinary skills in the art may make obvious modifications without departing from the principle and spirit of the present invention; however, all of such modifications shall be deemed as falling into the scope of protection of the present invention as defined by the claims.

1. A fuzzy diagnosis method for transformer internal composite defect based on gas dissolved in oil, comprising the following steps:
(I) acquiring monitoring data of volumetric concentrations of five types of monitored characteristic gases, i.e., hydrogen, methane, ethane, ethylene and acetylene; calculating the sum of the volumetric concentrations of methane, ethane, ethylene and acetylene (i.e., volumetric concentration of total hydrocarbons) from the monitoring data; judging whether the monitoring data of the five types of characteristic gases or the volumetric concentration of total hydrocarbons exceeds an alert value, which is selected as per the Chinese Standard GB/T7252-2001; if the monitoring data or the volumetric concentration of total hydrocarbons exceeds the alert value, further diagnosis is required; in that case, going to step (II); otherwise judging that the transformer has no defect or fault, if the monitoring data and the volumetric concentration of total hydrocarbons are normal;
(II) determining ratio codes:
first, the ratios are set as follows:
$${r}_{1} = \frac{{c}_{1} ( {C}_{2} {H}_{2} )}{{c}_{2} ( {C}_{2} {H}_{4} )} , \\ {r}_{2} = \frac{{c}_{3} ( {CH}_{4} )}{{c}_{4} ( {H}_{2} )} , \\ {r}_{3} = \frac{{c}_{2} ( {C}_{2} {H}_{4} )}{{c}_{5} ( {C}_{2} {H}_{6} )} , \\ {r}_{4} = \frac{{c}_{1} ( {C}_{2} {H}_{2} )}{{c}_{5} ( {C}_{2} {H}_{6} )} \times \frac{{c}_{2} ( {C}_{2} {H}_{4} )}{{c}_{1} ( {C}_{2} {H}_{2} )} = {r}_{3} \times \frac{{c}_{2} ( {C}_{2} {H}_{4} )}{{c}_{1} ( {C}_{2} {H}_{2} )} ,$$
wherein, c₁(C₂H₂), c₂(C₂H₄), c₃(CH₄), c₄(H₂) and c₅(C₂H₆) respectively represent the volumetric concentration of five types of characteristic gases (acetylene, ethylene, methane, hydrogen and ethane), in unit of μL/L;
then, the ratio codes are determined according to the following rules:
if r₁<0.1, the ratio code of r₁ is 0; if 0.15_r₁<1, the ratio code of r₁ is 1; if 5.1r₁<3, the ratio code of r₁ is 1; if r₁ the ratio code of r₁ is 2;
if r₂<0.1, the ratio code of r₂ is 1; if 0.1≦r₂<1, the ratio code of r₂ is 0; if 15-r₂<3, the ratio code of r₂ is 2; if the ratio code of r₂ is 2;
if r₃<0.1, the ratio code of r₃ is 0; if 0.15r₃<1, the ratio code of r₃ is 0; if 1.5r₃<3, the ratio code of r₃ is 1; if r₃≧3, the ratio code of r₃ is 2;
if r₄≦1.5, the ratio code of r₄ is 0; if r₄>1.5, the ratio code of r₄ is 1;
(III) Correcting the method for determining the types of transformer defects or faults on the basis of three ratios as specified in the Chinese Standard GB/T7252-2001:
based on the types of transformer defects or faults corresponding to the three ratio codes specified in the Chinese Standard GB/T 7252-2001, a ratio code 011 corresponding to the type of partial discharge defect or fault is added;
a fourth ratio r₄ is added on the basis of the three ratio codes; for the type of defect or fault with ratio code 101 diagnosed with the three-ratio method, if the transformer is judged as having a spark discharge defect or fault; if r₄>1.5, the transformer is judged as having an arc discharge defect or fault;
thus, obtaining a method for judging the types of transformer defects or faults according to ratio codes as follows:
if the ratio code of r₁ is 0, the ratio code of r₂ is 1, the ratio code of r₃ is 0, 1 or 2, and the ratio code of r₄ is 0 or 1, the type of transformer defect or fault is partial discharge;
if the ratio code of r₁ is 0, the ratio code of r₂ is 0, the ratio code of r₃ is 1, and the ratio code of r₄ is 0 or 1, the type of transformer defect or fault is low-temperature overheat lower than 300□;
if the ratio code of r₁ is 0, the ratio code of r₂ is 2, the ratio code of r₃ is 0, and the ratio code of r₄ is 0 or 1, the type of transformer defect or fault is low-temperature overheat lower than 300□;
if the ratio code of r₁ is 0, the ratio code of r₂ is 2, the ratio code of r₃ is 1, and the ratio code of r₄ is 0 or 1, the type of transformer defect or fault is 300-700° C. moderate-temperature overheat;
if the ratio code of r₁ is 0, the ratio code of r₂ is 0 or 2, the ratio code of r₃ is 2, and the ratio code of r₄ is 0 or 1, the type of transformer defect or fault is high-temperature overheat higher than 700□;
if the ratio code of r₁ is 2, the ratio code of r₂ is 0, 1 or 2, the ratio code of r₃ is 0, 1 or 2, and the ratio code of r₄ is 0 or 1, the type of transformer defect or fault is spark discharge;
if the ratio code of r₁ is 1, the ratio code of r₂ is 0, the ratio code of r₃ is 1, and the ratio code of r₄ is 0, the type of transformer defect or fault is spark discharge;
if the ratio code of r₁ is 1, the ratio code of r₂ is 0, the ratio code of r₃ is 1, and the ratio code of r₄ is 1, the type of transformer defect or fault is arc discharge;
if the ratio code of r₁ is 1, the ratio code of r₂ is 0, 1 or 2, the ratio code of r₃ is 0 or 2, and the ratio code of r₄ is 0 or 1, the type of transformer defect or fault is arc discharge;
if the ratio code of r₁ is 1, the ratio code of r₂ is 1 or 2, the ratio code of r₃ is 1, and the ratio code of r₄ is 0 or 1, the type of transformer defect or fault is arc discharge;
(IV)blurring the boundary ranges of the ratios r₁, r₂, r₃ and r₄ with a semi-Cauchy rising/falling function, and representing the rising edges and falling edges of the boundaries with the semi-Cauchy rising/falling function as follows:
$${μ}_{a} ( r ) = \{ \begin{matrix}1 , & r \leq A \\ \frac{1}{1 + {( \frac{A - r}{a} )}^{2}} , & others\end{matrix} \\ {μ}_{a} ( r ) = \{ \begin{matrix}1 , & r \geq A \\ \frac{1}{1 + {( \frac{A - r}{a} )}^{2}} , & others\end{matrix}$$
wherein, μ_{d}(r) is a falling edge function; μₐ(r) is a rising edge function; A is a boundary parameter; a is a distribution parameter; the values of A and a are as follows:
the rising edge boundary parameter of r₁ is 0.08, and the corresponding distribution parameter is 0.01, the falling edge boundary parameter of r₁ is 3.1, and the corresponding distribution parameter is 0.1;
the rising edge boundary parameter of r₂ is 0.06, and the corresponding distribution parameter is 0.02;
the falling edge boundary parameter of r₂ is 0.6, and the corresponding distribution parameter is 0.2;
the rising edge boundary parameter of r₃ is 0.8, and the corresponding distribution parameter is 0.1;
the falling edge boundary parameter of r₃ is 3.6, and the corresponding distribution parameter is 0.3;
the boundary parameter of r₄ is 1.43, and the corresponding distribution parameter is 0.1;
(V) obtaining the probabilities of the cases that the ratio codes of the ratios r₁, r₂ and r₃ are 0, 1 and 2 respectively and the probabilities of the cases that the ratio code of r₄ is 0 or 1 respectively, with the semi-Cauchy rising/falling function; the expressions are as follows:
Probability f-code0(r₁) of the case that the ratio code of r₁ is 0:
$$f - code0 ( {r}_{1} ) = \{ \begin{matrix}1 & ( {r}_{1} \leq 0.08 ) \\ \frac{1}{1 + {( \frac{0.08 - {r}_{1}}{0.01} )}^{2}} & ( {r}_{1} > 0.08 )\end{matrix}$$
Probability f-code1(r₁) of the case that the ratio code of r₁ is 1:
$$f - code 1 ( {r}_{1} ) = \{ \begin{matrix}\frac{1}{1 + {( \frac{0.08 - {r}_{1}}{0.01} )}^{2}} & ( {r}_{1} \leq 0.08 ) \\ 1 & ( 0.08 \leq {r}_{1} \leq 3.1 ) \\ \frac{1}{1 + {( \frac{3.1 - {r}_{1}}{0.1} )}^{2}} & ( {r}_{1} > 3.1 )\end{matrix}$$
probability f-code2(r₁) of the case that the ratio code of r₁ is 2:
$$f - code 2 ( {r}_{1} ) = \{ \begin{matrix}\frac{1}{1 + {( \frac{3.1 - {r}_{1}}{0.1} )}^{2}} & ( {r}_{1} < 3.1 ) \\ 1 & ( {r}_{1} \geq 3.1 )\end{matrix}$$
probability f-code0(r₂) of the case that the ratio code of r₂ is 0:
$$f - code 0 ( {r}_{2} ) = \{ \begin{matrix}\frac{1}{1 + {( \frac{0.06 - {r}_{2}}{0.02} )}^{2}} & ( {r}_{2} < 0.06 ) \\ 1 & ( 0.06 \leq {r}_{2} \leq 0.6 ) \\ \frac{1}{1 + {( \frac{0.06 - {r}_{2}}{0.02} )}^{2}} & ( {r}_{2} > 0.6 )\end{matrix}$$
probability f-code1(r₂) of the case that the ratio code of r₂ is 1:
$$f - code 1 ( r 2 ) = \{ \begin{matrix}1 & ( {r}_{2} \leq 0.06 ) \\ 1 + {( \frac{0.06 - {r}_{2}}{0.02} )}^{2} & ( {r}_{2} > 0.06 )\end{matrix}$$
probability f-code2(r₂) of the case that the ratio code of r₂ is 2:
$$f - code 2 ( {r}_{2} ) = \{ \begin{matrix}\frac{1}{1 + {( \frac{0.6 - {r}_{2}}{0.2} )}^{2}} & ( {r}_{2} < 0.6 ) \\ 1 & ( {r}_{2} \geq 0.6 )\end{matrix}$$
probability f-code0(r₃) of the case that the ratio code of r₃ is 0:
$$f - code 0 ( {r}_{3} ) = \{ \begin{matrix}1 & ( {r}_{3} \leq 0.8 ) \\ \frac{1}{1 + {( \frac{0.8 - {r}_{3}}{0.1} )}^{2}} & ( {r}_{3} > 0.8 )\end{matrix}$$
probability f-code1(r₃) of the case that the ratio code of r₃ is 1:
$$f - code 1 ( {r}_{3} ) = \{ \begin{matrix}\frac{1}{1 + {( \frac{0.6 - {r}_{3}}{0.1} )}^{2}} & ( {r}_{3} < 0.8 ) \\ 1 & ( 0.8 \leq {r}_{3} \leq 3.6 ) \\ \frac{1}{1 + {( \frac{3.6 - {r}_{3}}{0.3} )}^{2}} & ( {r}_{3} > 3.6 )\end{matrix}$$
probability f-code2(r₃) of the case that the ratio code of r₃ is 2:
$$f - code 2 ( {r}_{3} ) = \{ \begin{matrix}\frac{1}{1 + {( \frac{3.6 - {r}_{3}}{0.3} )}^{2}} & ( {r}_{3} > 3.6 ) \\ 1 & ( 0.8 \leq {r}_{3} \leq 3.6 )\end{matrix}$$
probability f-code0(r₄) of the case that the ratio code of r₄ is 0:
$$f - code 0 ( {r}_{4} ) = \{ \begin{matrix}1 & ( {r}_{4} \leq 1.43 ) \\ \frac{1}{1 + {( \frac{1.43 - {r}_{4}}{0.1} )}^{2}} & ( {r}_{4} > 1.43 )\end{matrix}$$
Probability f-code1(r₄) of the case that the ratio code of r₄ is 1:
$$f - code 1 ( {r}_{4} ) = \{ \begin{matrix}\frac{1}{1 + {( \frac{1.43 - {r}_{4}}{0.1} )}^{2}} & ( {r}_{4} < 1.43 ) \\ 1 & ( {r}_{4} \geq 1.43 )\end{matrix}$$
(VI)representing the probabilities of ratio codes with maximum value logic and minimum value logic, and thereby obtaining a fuzzy multi-value form of the diagnostic result of the types of transformer defects or faults; the probabilities of the types of transformer defects or faults are as follows:
f(partial discharge)=min[f-code0(r₁), f-code1(r₂)];
f(low-temperature overheat)=max{min[f-code0(r₁), f-code0(r₂), f-code1(r₃)], min[f-code0(r₁),
f-code2(r₂), f-code0(r3)]};
f(moderate-temperature overheat)=min[f-code0(r₁), f-code2(r₂), f-code1(r3)];
f(high-temperature overheat)=max{min[f-code0(r₁), f-code0(r₂), f-code2(r₃)], min[f-code0(r₁), f-code2(r2), f-code2(r₃)]};
f(spark discharge)=max{f-code2(r₁), min[f-code 1(r₁), f-code0(r₂), f-code1(r₃), f-code0(r₄)]};
f(arc discharge)=max{min[f-code 1(r₁), f-code0(r₂), f-code1(r₃), f-code1(r₄)], min[f-code1(r₁), f-code0(r₃)], min[f-code 1(r₁), f-code2(r₃)], min [f-code1(r₁), f-code1(r₂), f-code1(r₃)], min[f-code 1(r₁), f-code2(r₂), f-code 1(r₃)]}.
